SEQUENCE LISTING (2 sequences)

SEQ ID NO: 1 - protein, 40 aa, Artificial sequence
  Synthetic construct
GFGCNGPWNEDDLRCHNHCKSIKGYKGGYCAKGGFVCKCY

SEQ ID NO: 2 - protein, 40 aa, Pseudoplectania nigrella
  mat_peptide (1)..(40)
GFGCNGPWDEDDMQCHNHCKSIKGYKGGYCAKGGFVCKCY

(19) United States
(12) Patent Application Publication
(10) Pub. No.: US 2010/0104552 A1
(43) Pub. Date: Apr. 29, 2010

(54) ANTIBIOTIC SYNERGISM

(75) Inventors: Per Holse Mygind, Vaerloese (DK); Dorthe Sandvang, Slangerup (DK); Hans-Henrik Kristensen Hoegenhaug, Holte (DK); Thomas Kruse, Copenhagen (DK); Line Anker Nielsen, Copenhagen (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/604,440

(22) Filed: Oct. 23, 2009

Related U.S. Application Data
(60) Provisional application No. 61/112,358, filed on Nov. 7, 2008.
(60) Provisional application No. 61/118,782, filed on Dec. 1, 2008.
(60) Provisional application No. 61/157,630, filed on Mar. 5, 2009.

(30) Foreign Application Priority Data
Oct. 23, 2008 (EP) 08167451.7
Nov. 28, 2008 (EP) 08170260.7
Feb. 27, 2009 (EP) 09153967.6

(51) Int. Cl. A61K 38/00; A61K 31/43; A61K 31/545; A61P 31/04

(52) U.S. Cl. 424/114

(57) ABSTRACT

The present invention relates to antibiotic synergism of pharmaceutical compositions comprising a defensin and a beta-lactam antibiotic.

### CROSS-REFERENCE TO RELATED APPLICATIONS

This application claims priority or the benefit under 35 U.S.C. 119 of European application nos. EP 08167451.7, EP 08170260.7, and EP 09153967.6 filed Oct. 23, 2008, Nov. 28, 2008 and Feb. 27, 2009, respectively, and U.S. provisional application NOS. 61/112,358, U.S. 61/118,782 and 61/157,630 filed Nov. 7, 2008, Dec. 1, 2008 and Mar. 5, 2009, respectively, the contents of which are fully incorporated herein by reference.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to synergism between defensin antibiotics and beta-lactam antibiotics.

2. Description of Related Art

The few available distinct classes of antimicrobial compounds limit the scope for single and combination drug treatment of bacterial infections, including infections involving antibiotic-resistant bacteria. Antibacterial chemotherapy research has therefore focused on the discovery of novel targets for new antibacterial development. An alternative approach to the discovery of new antibacterial compounds is the discovery of antibiotic synergists. Synergism in antimicrobial therapy is well known and is used to describe supra-additive activity of antibiotics used in combinations. For example, in the treatment of bacterial infections combinations such as penicillin or ampicillin and streptomycin or gentamycin have been shown to have a supra-additive effect against enterococci infections. Similarly, carbenicillin or ticarcillin combined with an aminoglycoside such as gentamycin or tobramycin exhibit a synergistic effect in the treatment of Pseudomonas aeruginosa infection. Combined therapy using streptomycin together with tetracycline is more effective in the therapy of brucellosis than either agent alone, and a mixture of chloramphenicol plus a sulfonamide is more effective against meningitis due to Haemophilus influenzae.

Several antibacterial defensin polypeptides are known in the art. Examples include Plectasin (see WO 03/044049) and variants of Plectasin (see WO 2006/131504). Beta-lactam antibiotics are also a well-known group of antibacterial compounds. A beta-lactam (13-lactam) is a lactam with a heteroatomic ring structure, consisting of three carbon atoms and one nitrogen atom. The beta-lactam ring is part of several antibiotics, such as penicillin, which are therefore also called beta-lactam antibiotics.

Combinations of defensins and beta-lactam antibiotics, and therapeutic uses thereof, have not previously been disclosed.

It is an object of the present invention to provide a synergistic combination therapy of using defensins and beta-lactam antibiotics.

### SUMMARY OF THE INVENTION

We have now found that defensin polypeptides and beta-lactam antibiotics exhibit synergistic antibacterial activity.

Accordingly, the present invention provides a (synergistic) pharmaceutical composition comprising a first antibacterial compound which is a defensin, and a second antibacterial compound which is a beta-lactam antibiotic or an aminoglycoside.

In a second aspect, the invention provides a method for the treatment of a bacterial infection in a human or animal, comprising administering to the human or animal in need of such treatment a first antibacterial compound which is a defensin, and a second antibacterial compound which is a beta-lactam antibiotic or an aminoglycoside, in an effective amount for the treatment of the bacterial infection.

### DETAILED DESCRIPTION OF THE INVENTION

### Definitions

Antibacterial activity: The term “antibacterial activity” is defined herein as an activity which is capable of killing or inhibiting growth of bacterial cells. In the context of the present invention the term “antibacterial” is intended to mean that there is a bactericidal and/or a bacteriostatic effect; wherein the term “bactericidal” is to be understood as capable of killing bacterial cells; and wherein the term “bacteriostatic” is to be understood as capable of inhibiting bacterial growth. When growth of bacterial cells is inhibited, the cells are in a non-growing state, i.e., they are not able to propagate.

In a preferred embodiment, the term “antibacterial activity” is defined as bactericidal and/or bacteriostatic activity against Streptococci, preferably Streptococcus pneumoniae, or Staphylococci, preferably Staphylococcus aureus.

For purposes of the present invention, antibacterial activity may be determined according to the procedure described by Lehrer et al., 1991, Journal of Immunological Methods 137(2): 167-174. Alternatively, antibacterial activity may be determined according to the NCCLS guidelines from CLSI (Clinical and Laboratory Standards Institute; formerly known as National Committee for Clinical and Laboratory Standards).

Compounds having antibacterial activity may be capable of reducing the number of living cells of Streptococcus pneumoniae (ATCC 49619) to 1/100 after 24 hours (preferably after 16 hours, more preferably after 8 hours, most preferably after 4 hour, and in particular after 2 hours) incubation at 37° C. in a relevant microbial growth substrate at a concentration of 500 micrograms/mL; preferably at a concentration of 250 micrograms/mL; more preferably at a concentration of 100 micrograms/mL; even more preferably at a concentration of 50 micrograms/mL; most preferably at a concentration of 25 micrograms/mL; and in particular at a concentration of 10 micrograms/mL of the polypeptides having antimicrobial activity.

Compounds having antibacterial activity may also be capable of inhibiting the outgrowth of Streptococcus pneumoniae (ATCC 49619) for 8 hours at 37° C. in a relevant microbial growth substrate, when added in a concentration of 500 micrograms/mL; preferably when added in a concentration of 250 micrograms/mL; more preferably when added in a concentration of 100 micrograms/mL; even more preferably when added in a concentration of 50 micrograms/mL; most preferably when added in a concentration of 10 micrograms/mL; and in particular when added in a concentration of 5 micrograms/mL.

The compounds of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the antibacterial activity of the polypeptide consisting of the amino acid sequence of SEQ ID NO: 1.

Defensin: The term “defensin” as used herein refers to polypeptides recognized by a person skilled in the art as belonging to the defensin class of antimicrobial peptides. To determine if a polypeptide is a defensin according to the invention, the amino acid sequence is preferably compared with the hidden markov model profiles (HMM profiles) of the PFAM database by using the freely available HMMER software package (see Example 5).

The PFAM defensin families include Defensin_{—}1 or “Mammalian defensin” (accession no. PF00323), Defensin_{—}2 or “Arthropod defensin” (accession no. PF01097), Defensin_beta or “Beta Defensin” (accession no. PF00711), Defensin_propep or “Defensin propeptide” (accession no. PF00879) and Gamma-thionin or “Gamma-thionins family” (accession no. PF00304).

The defensins may belong to the alpha-defensin class, the beta-defensin class, the theta-defensin class, the insect or arthropod defensin classes, or the plant defensin class. The defensins from each of these classes share common structural features, such as the cysteine pattern. But it is important to note that the class affiliation does not reveal the source of the defensins. For example, a defensin from a fungus may be affiliated to the insect defensin class.

The defensin shown as SEQ ID NO: 1 is a synthetic defensin derived from Plectasin (see WO 03/044049), which is affiliated with the insect defensin class.

In an embodiment, the amino acid sequence of a defensin according to the invention comprises 4, 5, 6, 7, or 8 cysteine residues, preferably 4, 5, or 6 cysteine residues, more preferably 4 or 6 cysteine residues, and most preferably 6 cysteine residues.

The defensins may also be synthetic defensins sharing the characteristic features of any of the defensin classes.

Examples of such defensins include, but are not limited to, a-Defensin HNP-1 (human neutrophil peptide) HNP-2 and HNP-3; β-Defensin-12, Drosomycin, Heliomicin, γ1-purothionin, Insect defensin A, and the defensins disclosed in PCT applications WO 99/53053, WO 02/06324, WO 02/085934, WO 03/044049, WO 2006/131504, WO 2006/050737 and WO 2006/053565.

Isolated polypeptide: The term “isolated variant” or “isolated polypeptide” as used herein refers to a variant or a polypeptide that is isolated from a source. In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form. This can be accomplished, for example, by preparing the polypeptide by well-known recombinant methods or by classical purification methods.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides×100)/(Length of Alignment−Total Number of Gaps in Alignment).

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Modification: The term “modification” means herein any chemical modification of the polypeptide consisting of the amino acid sequence of SEQ ID NO: 1 as well as genetic manipulation of the DNA encoding that polypeptide. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus.

### Defensin Polypeptides Having Antibacterial Activity

In a first aspect, the present invention relates to isolated polypeptides having an amino acid sequence which has a degree of identity to SEQ ID NO: 1 (i.e., the mature polypeptides) of at least 80%, preferably at least 85%, more preferably at least 90%, most preferably at least 95%, and in particular at least 97%, which have antibacterial activity (hereinafter “homologous polypeptides”).

In a preferred aspect, the homologous polypeptides have an amino acid sequence which differs by at the most six amino acids, preferably by at the most five amino acids, more preferably by at the most four amino acids, even more preferably by at the most three amino acids, most preferably by at the most two amino acids, and in particular by one amino acid from the amino acid sequence of SEQ ID NO: 1.

In another preferred aspect, the homologous polypeptides have an amino acid sequence which differs by at the most six amino acids, preferably by at the most five amino acids, more preferably by at the most four amino acids, even more preferably by at the most three amino acids, most preferably by at the most two amino acids, and in particular by one amino acid from the amino acid sequence of SEQ ID NO: 2.

The amino acid sequences of SEQ ID NO: 1 and SEQ ID NO: 2 have three amino acid differences, which are in positions 9, 13 and 14.

A polypeptide of the present invention preferably comprises the amino acid sequence of SEQ ID NO: 1 or an allelic variant thereof. In a preferred aspect, a polypeptide comprises the amino acid sequence of SEQ ID NO: 1. In another preferred aspect, a polypeptide consists of the amino acid sequence of SEQ ID NO: 1 or an allelic variant thereof. In another preferred aspect, a polypeptide consists of the amino acid sequence of SEQ ID NO: 1.

Preferably, amino acid changes are of a minor nature, that is conservative amino acid substitutions or insertions that do not significantly affect the folding and/or activity of the polypeptide; single deletions; small amino- or carboxyl-terminal extensions; a small linker peptide of up to about 20-25 residues; or a small extension that facilitates purification by changing net charge or another function, such as a poly-histidine tag, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., antimicrobial activity) to identify amino acid residues that are critical to the activity of the molecule. See also, Hilton et al., 1996, J. Biol. Chem. 271: 4699-4708. The biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

### N-Terminal Extension

An N-terminal extension of the polypeptides of the invention may suitably consist of from 1 to 50 amino acids, preferably 2-20 amino acids, especially 3-15 amino acids. In one embodiment N-terminal peptide extension does not contain an Arg (R). In another embodiment the N-terminal extension comprises a kex2 or kex2-like cleavage site as will be defined further below. In a preferred embodiment the N-terminal extension is a peptide, comprising at least two Glu (E) and/or Asp (D) amino acid residues, such as an N-terminal extension comprising one of the following sequences: EAE, EE, DE and DD.

### Kex2 Sites

Kex2 sites (see, e.g., Methods in Enzymology, Vol 185, ed. D. Goeddel, Academic Press Inc. (1990), San Diego, Calif., “Gene Expression Technology”) and kex2-like sites are di-basic recognition sites (i.e., cleavage sites) found between the pro-peptide encoding region and the mature region of some proteins.

Insertion of a kex2 site or a kex2-like site have in certain cases been shown to improve correct endopeptidase processing at the pro-peptide cleavage site resulting in increased protein secretion levels.

In the context of the invention insertion of a kex2 or kex2-like site result in the possibility to obtain cleavage at a certain position in the N-terminal extension resulting in an antimicrobial polypeptide being extended in comparison to the mature polypeptide shown in SEQ ID NO: 1.

### Fused Polypeptides

The polypeptides of the present invention also include fused polypeptides or cleavable fusion polypeptides in which another polypeptide is fused at the N-terminus or the C-terminus of the polypeptide of the invention or a fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

### Mechanism-Of-Action of the Defensins Shown as SEQ ID NO: 1 and SEQ ID NO: 2

The mechanism by which the defensins shown as SEQ ID NO: 1 (hereinafter “Defensin2114”) and SEQ ID NO: 2 (hereinafter “Plectasin”) kills bacterial cells has been thoroughly investigated using a number of techniques. In contrast to many other antimicrobial peptides, Defensin2114 and Plectasin do not initially and as a main mechanism compromise the bacterial membrane. No K⁺-efflux could be detected and the trans-membrane potential of bacteria exposed to Defensin2114 or Plectasin remained intact.

Traditional incorporation studies showed that exposure to Defensin2114 or Plectasin severely affected incorporation of cell wall precursors but not those of proteins or nucleic acids (RNA/DNA).

It has been shown in complementing experiments such as DNA microarray, growth kinetics and changes in cellular morphology that the result of Defensin2114-exposure or Plectasin-exposure mimics the response after exposure to other cell-wall inhibiting agents and mostly that of vancomycin. More specifically, it has been shown in vitro that Defensin2114 and Plectasin binds to LipidI and LipidII—precursors used to synthesize the bacterial cell wall—with binding-constants in the 10⁻⁶ to 10⁻⁷ M⁻¹ range. Sequestering of LipidII (and LipidI) in the bacterial cell inhibits the polymerization of these precursors into peptidoglycan. The polymerization of LipidI into peptidoglycan is a result of an initial trans-glycosylation of the sugar-moieties followed by a transpeptidase reaction where the cross-linking of different sugar-strands is mediated through the peptide part of LipidII. Both reactions could in principle be blocked by Defensin2114 or Plectasin.

Other antibiotics and antimicrobials have also been shown to bind and sequester LipidII, e.g., the glycopeptide vancomycin and the Iantibiotic Nisin. No cross-resistance have been observed between, e.g., vancomycin-resistant Staphylococcus aureus (VRSA) and Defensin2114 or Plectasin indicating that different epitopes on LipidII is involved in/important for binding of these two antibiotics.

### Mechanism-Of-Action of Beta-Lactam Antibiotics

The beta-lactam ring is part of the structure of several antibiotic families, principally the penicillins, cephalosporins, carbapenems and monobactams, which are therefore also called beta-lactam antibiotics. These antibiotics work by inhibiting the bacterial cell wall synthesis. Beta-lactam antibiotics (such as Penicillin G) work by inhibiting the formation of peptidoglycan cross-links in the bacterial cell wall. The β-lactam moiety (functional group) of penicillin binds to the enzyme (DD-transpeptidase) that links the peptidoglycan molecules in bacteria, which weakens the cell wall of the bacterium (in other words, the antibiotic causes cytolysis or death due to osmotic pressure). In addition, the build-up of peptidoglycan precursors triggers the activation of bacterial cell wall hydrolases and autolysins, which further digest the bacteria's existing peptidoglycan.

Penicillin shows a synergistic effect with aminoglycosides (such as gentamicin), since the inhibition of peptidoglycan synthesis allows aminoglycosides to penetrate the bacterial cell wall more easily, allowing its disruption of bacterial protein synthesis within the cell. This results in a lowered MBC for susceptible organisms.

Cephalosporin compounds were first isolated from cultures of Cephalosporium acremonium from a sewer in Sardinia in 1948 by Italian scientist Giuseppe Brotzu. The cephalosporin nucleus, 7-aminocephalosporanic acid (7-ACA), was derived from cephalosporin C and proved to be analogous to the penicillin nucleus 6-aminopenicillanic acid. Therefore, cephalosporin compounds belong to the group of beta-lactam antibiotics.

Cephalosporins (such as Ceftriaxone or Cefotaxime) are bactericidal and have the same mode of action as other beta-lactam antibiotics (such as penicillins). Cephalosporins disrupt the synthesis of the peptidoglycan layer of bacterial cell walls. The peptidoglycan layer is important for cell wall structural integrity. The final transpeptidation step in the synthesis of the peptidoglycan is facilitated by transpeptidases known as penicillin binding proteins (PBPs). PBPs bind to the D-Ala-D-Ala at the end of muropeptides (peptidoglycan precursors) to crosslink the peptidoglycan. Beta-lactam antibiotics mimic this site and competitively inhibit PBP crosslinking of peptidoglycan.

### Mechanism of Action of Aminoglycosides

Aminoglycosides (such as Gentamicin) work by binding to the bacterial 30S ribosomal subunit (some work by binding to the 50S subunit), inhibiting the translocation of the peptidyl-tRNA from the A-site to the P-site and also causing misreading of mRNA, leaving the bacterium unable to synthesize proteins vital to its growth. They kill bacteria by inhibiting protein synthesis as they bind to the 16S rRNA and by disrupting the integrity of bacterial cell membrane.

### Compositions Exhibiting Synergistic Antibacterial Activity

The present invention provides a pharmaceutical composition, exhibiting synergistic antibacterial activity, comprising:
a first antibacterial compound which is a defensin, and
a second antibacterial compound which is a beta-lactam antibiotic or an aminoglycoside.

Synergism is identified in two-dimensional or checkerboard tests when the Fractional Inhibitory Concentration (FIC) index is ≦0.5. In killing curves, synergy is identified as a ≧2 Log₁₀, decrease in CFU/mL between the combination and the most active constituent after 24 hours; and the number of surviving organisms in the presence of the combination must be ≧2 Log₁₀ CFU/mL below the starting inoculum. At least one of the drugs must be present in a concentration which does not affect the growth curve of the test organism, when used alone. Antagonism is identified by a FIC index ≧4. See also Examples 1 and 2.

In an embodiment, the composition of the invention exhibits a FIC index ≦0.5 using a test organism selected from the group consisting of Streptococcus pneumoniae ATCC49619, Staphylococcus aureus ATCC29213, Staphylococcus aureus ATCC34400, Staphylococcus aureus ATCC25923, and Staphylococcus epidermidis ATCC49134.

In another embodiment, the composition of the invention exhibits synergistic antibacterial activity on Gram-positive bacteria, such as streptococci and staphylococci.

### Methods and Uses

The composition of the invention is used for treating bacterial infections. Accordingly, the composition of the invention may be used as an antibacterial veterinarian or human therapeutic or prophylactic agent. The composition of the invention may be used in the preparation of veterinarian or human therapeutic agents or prophylactic agents for the treatment of bacterial infections.

The composition of the invention is used in an amount sufficient to treat the bacterial infection, i.e., sufficient to kill or inhibit the bacteria causing the bacterial infection; for example, to kill or inhibit growth of Streptococcus sp., such as Streptococcus pneumoniae, or Staphylococcus sp., such as Staphylococcus aureus.

Formulations of the composition of the invention are administered to a host suffering from a bacterial infection. Administration may be localized or systemic. Generally the dose of the composition of the invention will be sufficient to decrease the bacterial population by at least 1 log, and may be by 2 or more logs of killing. The composition of the invention is administered at a dosage that reduces the bacterial population while minimizing any side-effects. It is contemplated that the composition will be obtained and used under the guidance of a physician for in vivo use.

The antibacterial compounds used in the composition of the invention may be administered simultaneously, or after each other, in any order. The antibacterial compounds may also be administered independently of each other via different routes. The following examples of administration and formulation apply to the composition of the invention, as well as to the individual antibacterial compounds used in the composition.

Various methods for administration may be employed. The formulation of the antibacterial compounds may be given orally, or may be injected intravascularly, intramuscular, subcutaneously, peritoneally, by aerosol, opthalmically, intra-bladder, topically, etc. The dosage of the therapeutic formulation will vary widely, depending on the specific antibacterial compounds to be administered, the frequency of administration, the manner of administration, the clearance of the agent from the host, and the like. The initial dose may be larger, followed by smaller maintenance doses. In many cases, oral administration will require a higher dose than if administered intravenously. The amide bonds of the defensin, as well as the amino and carboxy termini, may be modified for greater stability on oral administration. For example, the carboxy terminus may be amidated.

### Formulations

The antibacterial compounds used in the composition of this invention can be incorporated into a variety of formulations for therapeutic administration. More particularly, the antibacterial compounds can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the antibacterial compounds can be achieved in various ways, including oral, buccal, rectal, parenteral, intraperitoneal, intradermal, transdermal, intracheal, etc., administration. The antibacterial compounds of the composition of the invention may be systemic after administration or may be localized.

The composition of the present invention can be administered alone, or in combination with other known compounds (e.g., perforin, anti-inflammatory agents, antibiotics, etc.). In pharmaceutical dosage forms, the antibacterial compounds of the composition may be administered in the form of their pharmaceutically acceptable salts. The following methods and excipients are merely examples and are in no way limiting.

For oral preparations, the antibacterial compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The antibacterial compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The antibacterial compounds can be utilized in aerosol formulation to be administered via inhalation. The antibacterial compounds used in the composition of the invention can be formulated into pressurized acceptable propellants such as dichlorodifluoromethane, propane, nitrogen and the like.

Furthermore, the antibacterial compounds can be made into suppositories by mixing with a variety of bases such as emulsifying bases or water-soluble bases. The antibacterial compounds can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the antibacterial compounds used in the composition of the invention. Similarly, unit dosage forms for injection or intravenous administration may comprise the antibacterial compounds as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the composition of the invention is placed in proximity to the site of infection, so that the local concentration of the antibacterial compounds is increased relative to the rest of the body.

The term “unit dosage form”, as used herein, refers to physically discrete units suitable as unitary dosages for human and animal subjects, each unit containing a predetermined quantity of antibacterial compounds of the present invention calculated in an amount sufficient to produce the desired effect in association with a pharmaceutically acceptable diluent, carrier or vehicle. The specifications for the unit dosage forms of the present invention depend on the particular antibacterial compounds employed and the effect to be achieved, and the pharmacodynamics associated with the antibacterial compounds in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific antibacterial compounds, the severity of the symptoms and the susceptibility of the subject to side effects. Some of the specific antibacterial compounds are more potent than others. Preferred dosages for a given antibacterial compound are readily determinable by those of skill in the art by a variety of means. A preferred means is to measure the physiological potency of a given antibacterial compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like.

For preparing the liposomes, the procedure described by Kato et al., 1991, J. Biol. Chem. 266:3361 may be used. Briefly, the lipids and lumen composition containing antibacterial compounds are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the antibacterial compounds used in the composition of the invention may be formulated with other pharmaceutically active agents, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the antibacterial compounds, e.g., interferon gamma, tumor necrosis factor alpha, interleukin 12, etc.

In vitro Synthesis

The defensin polypeptides used in the composition of the invention may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms), e.g., D-alanine and D-isoleucine, diastereoisomers, side chains having different lengths or functionalities, and the like. The particular sequence and the manner of preparation will be determined by convenience, economics, purity required, and the like.

Chemical linking may be provided to various peptides or proteins comprising convenient functionalities for bonding, such as amino groups for amide or substituted amine formation, e.g., reductive amination, thiol groups for thioether or disulfide formation, carboxyl groups for amide formation, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein

The present invention is further described by the following examples that should not be construed as limiting the scope of the invention.

### EXAMPLES

The defensin polypeptide shown as SEQ ID NO: 1 is hereinafter referred to as “Defensin2114”.

The defensin polypeptide shown as SEQ ID NO: 2 is hereinafter referred to as “Plectasin”.

### Example 1

### Antibiotic Synergism I

Synergy, additivity and antagonism between Defensin2114 and a variety of clinically employed antibiotics were investigated using a Time-Kill approach against Streptococcus pneumoniae ATCC49619, Staphylococcus aureus ATCC29213, Staphylococcus aureus ATCC34400, Staphylococcus aureus ATCC25923, and Staphylococcus epidermidis ATCC49134.

In this approach, individual antibiotics at ½×MIC were added to 10⁷ CFU/mL staphylococcal or streptococcal cells, either alone or in specific combinations, and the CFU was determined at 2, 3 and 5 hours after exposure.

Synergy was defined as a ≧2 log₁₀ decrease in CFU/ml for the combination compared with the most active single agent alone.

Additivity was defined as a ≧1 log₁₀ decrease with the combination in comparison with the most active single antimicrobial alone.

Antagonism was defined as a ≧2 log₁₀ increase in colony count. The antibiotics tested in combination with Defensin2114 were: Gentamicin, Penicillin G, Ceftriaxone, Vancomycin, Erythromycin, Ciprofloxacin, Tetracycline, Polymyxin B, Fucidin, Linezolid, Mupirocin and Choramphenicol.

Synergism was identified between Defensin2114 and Penicillin G, and between Defensin2114 and Ceftriaxone. Both Penicillin G and Ceftriaxone are beta-lactam antibiotic compounds that inhibit the transpeptidation step in the synthesis of the bacterial peptidoglycan. Defensin2114 and Gentamicin showed synergy at some but not all time-points.

Lack of antagonism was observed between Defensin2114 and all compounds tested (Gentamicin, Penicillin G, Ceftriaxone, Vancomycin, Erythromycin, Ciprofloxacin, Tetracycline, Polymyxin B, Fucidin, Linezolid, Mupirocin and Choramphenicol).

### Example 2

### Antibiotic Synergism II

Synergy, additivity and antagonism between Defensin2114 and a variety of clinically employed antibiotics was also investigated using a checkerboard titration assay against Staphylococcus aureus ATCC29213.

This approach can be described essentially as an MIC/MBC assay in 2 dimensions. In short, one antibiotic is serially 2-fold diluted along one direction of a microtiter plate while the second antibiotic is diluted along the other direction of the microtiter plate. In addition, each well contains a suspension of ˜5×10⁴ bacteria, and the plate is read after 18-24 hours of incubation at 37° C. The resulting growth or no growth is recorded and the Fractional Inhibitory Concentration (FIC) index is calculated as the minimal combination of the two compounds that inhibits growth on the microtiter plate following the formula below:

FIC index=(conc. of drug A/MIC of drug A)+(conc. of drug B/MIC of drug B)

Fractional Bactericidal Concentration (FBC) is calculated in a similar manner, but based on MBC.

Interpretation of results is (in accordance with the literature):

| | | |
|---|---|---|
| FBC/FIC Index | ≦0.5 | ~synergism |
| 0.5 < FBC/FIC Index | <4 | ~lack of synergy/antagonism |
| FBC/FIC Index | ≧4.0 | ~antagonism |

The antibiotics tested in combination with Defensin2114 included: Erythromycin, Penicillin G, Gentamicin, Ceftriaxone, Vancomycin, Ciprofloxacin and Linezolid.

The calculated FIC/FBC indices are shown in Table 1.

TABLE 1

| | | FIC | FBC |
|---|---|---|---|
| First antibiotic | Second antibiotic | index | index |
| Defensin2114 | Defensin2114 | 1 | — |
| Defensin2114 | Erythromycin | 1.5 | — |
| Defensin2114 | Penicillin G | 0.5 | 0.5 |
| Defensin2114 | Gentamicin | 1 | — |
| Defensin2114 | Ceftriaxone | 0.5 | — |
| Defensin2114 | Vancomycin | 2 | — |
| Defensin2114 | Ciprofloxacin | 1 | — |
| Defensin2114 | Linezolid | 2 | — |

As observed in the Time-Kill approach described in Example 1, synergism between Defensin2114 and Penicillin G or Ceftriaxone (FBC/FIC=0.5) was found against Staphylococcus aureus.

No FIC antagonism was found between Defensin2114 and Erythromycin, Gentamicin, Vancomycin, Ciprofloxacin and Linezolid.

### Example 3

### Antibiotic Synergism III

Synergy between Defensin2114 and clinically employed antibiotics, penicillin-G and ceftriaxone, was investigated using a checkerboard titration assay against representative species of Entorococci, Staphylococci and Streptococci. This approach can be described essentially as a MIC assay in two dimensions. In short, one antibiotic is serially two-fold diluted along one direction of the microtitre plate while the second antibiotic is diluted along the other direction. In addition, each well contains a suspension of ˜5×10⁴ bacteria and the plate is read after 18 hours of incubation at 37° C. The analysis results in a checkerboard of serial dilutions of both antibiotics. The Fractional Inhibitory Concentration (FIC) index is generally calculated as the minimal combination of the two compounds that inhibits growth on the microtitre plate. This value was calculated as:

FIC index=(Conc. of drug A/MIC of drug A)+(Conc. of drug B/MIC of drug B)

Interpretation of results was (in accordance with the literature):

| | | |
|---|---|---|
| FBC/FIC Index | ≦0.5 | ~synergism |
| 0.5 < FBC/FIC Index | <4 | ~lack of synergy/antagonism |
| FBC/FIC Index | ≧4.0 | ~antagonism |

The study resulted in FIC indices indicating antibiotic synergy of the following combinations of antibiotics, as described in Table 2 below.

TABLE 2

| | | | | FIC |
|---|---|---|---|---|
| 1. antibiotic | 2. antibiotic | Target strain | ATCC# | index |
| Defensin2114 | Ceftriaxone | Enterococcus faecalis | 29212 | 0.25 |
| Defensin2114 | Penicillin G | Staphylococcus aureus | 25923 | 0.5 |
| Defensin2114 | Ceftriaxone | Staphylococcus aureus | 25923 | 0.5 |
| Defensin2114 | Penicillin G | Staphylococcus aureus | 29213 | 0.5 |
| Defensin2114 | Ceftriaxone | Staphylococcus aureus | 29213 | 0.5 |
| Defensin2114 | Penicillin G | Staphylococcus aureus | 43300 | 0.25 |
| | | MRSA | | |
| Defensin2114 | Ceftriaxone | Staphylococcus aureus | 43300 | 0.25 |
| | | MRSA | | |
| Defensin2114 | Ceftriaxone | Streptococcus pyogenes | 12344 | 0.5 |

The results in Table 2 indicate synergy between Defensin2114 and ceftriaxone or penicillin against representative strains of Enterococci, Staphylococci and Streptococci. The results also show synergy between Defensin2114 and penicillin/ceftriaxzone against a methicillin resistant Staphylococcus aureus isolate.

### Example 4

### Antibiotic Synergism IV

Synergy between Plectasin and a variety of clinically employed antibiotics was investigated against Staphylococcus aureus ATCC29213, using the procedure described in Examples 2 and 3.

The antibiotics tested in combination with Plectasin were: Erythromycin, Penicillin G, Gentamicin, Vancomycin, Ciprofloxacin and Cefotaxime. Penicillin G and Cefotaxime are beta-lactam antibiotics. The calculated FIC indices are shown in Table 3.

TABLE 3

| | | FIC |
|---|---|---|
| First antibiotic | Second antibiotic | index |
| Plectasin | Erythromycin | 1.5 |
| Plectasin | Penicillin G | 0.5 |
| Plectasin | Gentamicin | 1.5 |
| Plectasin | Cefotaxime | 0.5 |
| Plectasin | Vancomycin | 2 |
| Plectasin | Ciprofloxacin | 0.75 |

Synergism between Plectasin and Penicillin G or Cefotaxime (FIC=0.5) was found against Staphylococcus aureus.

No FIC antagonism was found between Plectasin and Erythromycin, Gentamicin, Vancomycin or Ciprofloxacin.

### Example 5

### Using the HMM Files from the PFAM Database to Identify a Defensin

Sequence analysis using hidden markov model profiles (HMM profiles) may be carried out either online on the Internet or locally on a computer using the well-known HMMER freely available software package. The current version is HMMER 2.3.2 from October 2003.

The HMM profiles may be obtained from the well-known PFAM database. The current version is PFAM 16.0 from November 2004. Both HMMER and PFAM are available for all computer platforms from, e.g., Washington University in St. Louis (USA), School of Medicine (http://pfam.wustl.edu and http://hmmer.wustl.edu).

If a query amino acid sequence, or a fragment thereof, belongs to one of the following five PFAM families, the amino acid sequence is a defensin according to the present invention:

Defensin_beta or “Beta Defensin”, accession number: PF00711;

Defensin_propep or “Defensin propeptide”, accession number: PF00879;

Defensin 1 or “Mammalian defensin”, accession number: PF00323;

Defensin_{—}2 or “Arthropod defensin”, accession number: PF01097;

Gamma-thionin or “Gamma-thionins family”, accession number: PF00304.

An amino acid sequence belongs to a PFAM family, according to the present invention, if it generates an E-value which is greater than 0.1, and a score which is larger or equal to zero, when the PFAM database is used online, or when the hmmpfam program (from the HMMER software package) is used locally.

When the sequence analysis is carried out locally using the hmmpfam program, it is necessary to obtain (download) the HMM profiles from the PFAM database. Two profiles exist for each family; xxx_ls.hmm for glocal searches, and xxx_fs.hmm for local searches (“xxx” is the name of the family). That makes a total of ten profiles for the five families mentioned above.

These ten profiles may be used individually, or joined (appended) into a single profile (using a text editor—the profiles are ASCII files) that could be named, e.g., defensin.hmm. A query amino acid sequence can then be evaluated by using the following command line:

hmmpfam-E 0.1 defensin.hmm sequence_file

wherein “sequence_file” is a file with the query amino acid sequence in any of the formats recognized by the HMMER software package.

If the score is larger or equal to zero (0.0), and the E-value is greater than 0.1, the query amino acid sequence is a defensin according to the present invention.

The PFAM database is further described in Bateman et al., 2004, “The Pfam Protein Families Database”, Nucleic Acids Research 32 (Database Issue): D138-D141.

1. A pharmaceutical composition comprising:
(a) a first antibacterial compound which is a defensin, and
(b) a second antibacterial compound which is a beta-lactam antibiotic or an aminoglycoside.

2. The composition of claim 1, wherein the first and second antibacterial compound exhibit a synergistic antibacterial activity.

3. The composition of claim 2, wherein the first and second antibacterial compound exhibit a FIC index ≦0.5 using a test organism selected from the group consisting of Streptococcus pneumoniae ATCC49619, Staphylococcus aureus ATCC29213, Staphylococcus aureus ATCC34400, Staphylococcus aureus ATCC25923, and Staphylococcus epidermidis ATCC49134.

4. The composition of claim 1, wherein the first antibacterial compound binds to the bacterial cell wall precursor Lipid I and/or Lipid II.

5. The composition of claim 4, wherein the binding results in sequestering of Lipid I and/or Lipid II.

6. The composition of claim 4, wherein the binding is characterized by a binding constant of about 10⁻⁶ M⁻¹ or less, preferably by a binding constant of about 10⁻⁷ M⁻¹.

7. The composition of claim 1, wherein the first antibacterial compound belongs to the arthropod defensin class, or the insect defensin class.

8. The composition of claim 1, wherein the defensin comprises an amino acid sequence having at least 80% identity, preferably at least 85% identity, more preferably at least 90% identity, even more preferably at least 95% identity, and most preferably at least 97% identity to the amino acid sequence of SEQ ID NO: 1.

9. The composition of claim 1, wherein the second antibacterial compound is selected from the group consisting of penicillins, cephalosporins and aminoglycosides.

10. The composition of claim 1, wherein the second antibacterial compound is selected from the group consisting of Penicillin G, Ceftriaxone and Gentamicin.

11. A method for the treatment of an intracellular bacterial infection, comprising administering to a human or animal in need of such treatment a composition of claim 1 in an effective amount for the treatment of the intracellular bacterial infection.

12. The method of claim 11, wherein the bacterial infection is caused by Gram-positive bacteria.

13. The method of claim 12, wherein the bacterial infection is caused by Streptococci or Staphylococci.
